(19) United States
(12) Patent Application Publication
(10) Pub. No.: US 2009/0047228 A1
(43) Pub. Date: Feb. 19, 2009

(54) ASSEMBLY FOR CONDITIONING AND APPLYING A NAIL VARNISH OF HIGH VISCOSITY

(75) Inventors: Laurence GUERCHET, Chevilly-Larue (FR); Lizabeth-Anne Coffey-Dawe, Aulnay Sous Bois (FR); Aline Abergel, Boulogne (FR); Marc Ramet, Asnieres (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/188,279

(22) Filed: Aug. 8, 2008

Related U.S. Application Data
(60) Provisional application No. 60/956,938, filed on Aug. 21, 2007.
(60) Provisional application No. 60/956,937, filed on Aug. 21, 2007.

(30) Foreign Application Priority Data
Aug. 10, 2007 (FR) 07 05814
Aug. 10, 2007 (FR) 07 05815

(51) Int. Cl. A61K 8/00; A45D 34/04; A61Q 3/02

(52) U.S. Cl. 424/61; 401/11; 401/25; 401/130

(57) ABSTRACT

A conditioning and application assembly includes a container containing a nail varnish composition comprising a cosmetically acceptable medium and at least one thixotropic thickener, with the composition having a viscosity at 25° C. of at least 0.6 Pa·s. An applicator is provided for taking up the product contained in the container and applying it to the nails, with the applicator including a flexible blade defining an application face.

### REFERENCE TO PRIOR APPLICATIONS

This application claims priority to U.S. provisional application Nos. 60/956,937 and 60/956,938 both filed Aug. 21, 2007; and to French patent applications 0705815 and 0705814, both filed Aug. 10, 2007, all four incorporated herein by reference.

### FIELD OF THE INVENTION

The present invention relates to a conditioning and application assembly for applying a product of high viscosity to the nails. The present invention also relates to a high viscosity nail varnish of gelled texture preferably comprising particular coloring agents, and also to a process for coating the nails.

### BACKGROUND OF THE INVENTION

Conventional nail varnishes are in liquid or fluid form and are generally conditioned in bottles. They generally comprise solid particles such as pigments, nacres or fillers, which are in dispersion in the continuous aqueous medium or the organic solvent medium of the composition.

This fluid form imposes good dispersion of the pigments so as to preserve the homogeneity of the color of the liquid varnish and also of the film of varnish once applied to the nails.

However, these particles have a tendency to sediment over time, due to their density, which is higher than that of the continuous medium in which they are dispersed. This sedimentation results in a change in the microscopic appearance of the composition, and in particular, in the case of colored nail varnishes, in heterogeneity of the color of the varnish. The incorporation of this sort of particle, in particular of nacres and magnetic pigments, is thus limited (to a maximum of 1 to 2% depending on the sort of particle) in conventional liquid nail varnishes.

### SUMMARY OF THE INVENTION

It is thus sought to obtain nail varnishes that have a good dispersion of the particles, in particular of the pigments, and thus good stability and good homogeneity of the color over time, and also a film that has satisfactory covering power when applied to the nails. In addition, from a practical viewpoint, it would be advantageous to have available varnishes with novel textures that are easy to manipulate (no problems of spilling or dripping).

The inventors have found that these advantages are obtained by using a high-viscosity nail varnish composition in non-liquid form and of gelled texture, which allows uniform dispersion of the pigments when present. According to a preferred alternative, the pigments are present in an amount of more than or equal to 2% by weight of the composition.

Furthermore, this gelled texture allows better organization and orientation of the coloring particles (in particular of the nacres) in the composition when it is applied to the nails and then during the drying of the film of varnish, thus making it possible to obtain a color effect and gloss that are superior to those of films derived from conventional fluid nail varnishes in which the coloring particles do not follow a preferential orientation.

In addition, contrary to conventional nail varnishes, this composition does not flow, does not drip and makes it possible to obtain, after application to the nails, a film that dries quickly while at the same time being uniform and smooth; and that has good staying power and gloss properties.

Moreover, conventional nail varnishes and nailcare products are generally applied using fine brushes that are made by attaching a tuft of bristles to the end of a shaft. Such fine brushes are usually satisfactory for products of liquid consistency.

However, for more viscous products, conventional fine brushes do not give good results since the bristles form striations on the surface of the product, which remain on drying.

There is a need for varnishes with novel textures, of high viscosity and gelled texture, which are easy to manipulate (no problems of spilling or dripping), which show good particle dispersion, and thus good stability and good homogeneity of the color over time, and also from an applicator that is more particularly suited to the application of the varnish with a gel texture.

The colored or transparent nail varnish composition or product may be used as a varnish base or “base coat”, as a nail makeup product, as a finishing composition, also known as a “top coat”, to be applied over the nail makeup product, or alternatively as a nailcare product.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

As should be apparent, the invention can provide a number of advantageous features and benefits. It is to be understood that, in practicing the invention, an embodiment can be constructed to include one or more features or benefits of embodiments disclosed herein, but not others. Accordingly, it is to be understood that the preferred embodiments discussed herein are provided as examples and are not to be construed as limiting, particularly since embodiments can be formed to practice the invention that do not include each of the features of the disclosed examples.

### BRIEF DESCRIPTION OF THE DRAWINGS

The invention may be understood more clearly on reading the detailed description that follows, of non-limiting implementation examples thereof, and on examining the attached drawing, in which:

FIG. 1 is a schematic longitudinal section of an example of a conditioning and application assembly according to the invention,

FIG. 2 shows, in isolation and partially, the applicator viewed from the side,

FIG. 3 shows, in isolation and partially, the applicator viewed from above,

FIG. 4 is a cross section along IV-IV of FIG. 3,

FIG. 5 illustrates the uptake of product with the applicator,

FIG. 6 illustrates the application to the nail,

FIG. 7 illustrates the possibility that the user has of modifying the curvature of the application face,

FIG. 8 is a similar view to FIG. 4 of an embodiment variant,

FIG. 9 is a cross section of the applicator of FIG. 8 during application of product to the nail,

FIG. 10 is a similar view to FIG. 4 of an embodiment variant,

FIGS. 11 to 14 schematically show, in top view, other examples of applicators,

FIGS. 15 and 16 schematically show, in perspective, other examples of applicators,

FIGS. 17 to 20 show other examples of conditioning and application assemblies according to the invention,

FIG. 21 shows another example of an applicator,

FIG. 22 schematically shows, in perspective, an embodiment variant of the applicator,

FIG. 23 is a face view of the applicator of FIG. 22,

FIG. 24 is a side view of the applicator of FIG. 22,

FIG. 25 is a longitudinal section along XXV-XXV of FIG. 23,

FIG. 26 is an axial view of the applicator of FIG. 22 from the end of the flexible blade,

FIG. 27 shows, in top view, an embodiment variant of the applicator,

FIG. 28 is a bottom view of the applicator of FIG. 27,

FIG. 29 shows the applicator of FIG. 27, viewed from the side,

FIG. 30 is a section along XXX-XXX of FIG. 29,

FIG. 31 is a view in perspective of the applicator of FIG. 27, and

FIG. 32 partially shows the free edge of the flexible blade of an applicator embodiment variant.

### DETAILED DESCRIPTION OF THE PREFERRED EMBODIMENTS

According to a first of its aspects, one subject of the invention is thus a conditioning and application assembly comprising:
a container (3) containing a nail varnish composition (or product) comprising a cosmetically acceptable medium and at least one thixotropic thickener, the composition having a viscosity at 25° C. of at least 0.6 Pa·s,
an applicator (2) for taking up the product contained in the container and applying it to the nails, comprising a flexible blade (6) defining an application face (15).

According to another aspect, a subject of the present invention is a nail varnish composition comprising a cosmetically acceptable medium, at least one thixo-tropic thickener, at least one coloring agent chosen from magnetic substances, diffractive pigments, interference pigments, reflective particles, and mixtures thereof, the coloring agent being present in a content of greater than or equal to 2% by weight relative to the total weight of the composition, the composition having a viscosity at 25° C. of at least 0.6 Pa·s.

According to a preferred example, the nature and/or amount of the thickener is (are) such that, in response to a non-chemical action, especially a mechanical action, prior to or simultaneously with the application of the composition to the nails, the viscosity of the composition may be reversibly lowered to a value not exceeding 0.4 Pa·s and preferably to value not exceeding 0.3 Pa·s.

According to another aspect, a subject of the invention is a conditioning and application assembly comprising:
a container (3) containing a nail varnish composition (or product) comprising a cosmetically acceptable medium, at least one thixotropic thickener, at least one coloring agent chosen from magnetic substances, diffractive pigments, interference pigments and reflective particles, and mixtures thereof, the coloring agent being present in a content of greater than or equal to 2% by weight relative to the total weight of the composition, the composition having a viscosity at 25° C. of at least 0.6 Pa·s, and

an applicator (2) for taking up the product contained in the container and applying it to the nails, comprising a flexible blade (6) defining an application face (15).

Preferably, the compositions according to the invention are free of camphor. The term “free of” means that the composition contains no camphor at all, i.e. an amount of 0% by weight relative to the total weight of the composition.

### Measurement of the Viscosity

The viscosity of the composition is measured at 25° C. using a Rheomat 180 viscometer (from the company Lamy) equipped with an MS-R1, MS-R2, MS-R3, MS-R4 or MS-R5 spindle chosen as a function of the consistency of the composition, rotating at a spin speed of 200 rpm. The measurement is taken after 10 minutes of rotation. The viscosity measurements are performed not more than one week after manufacture.

The nail varnish of the invention may preferably have a viscosity ranging for example from 0.6 to 20 Pa·s, preferably from 0.7 to 15 Pa·s and better still from 0.75 to 10 Pa·s.

A subject of the invention is also a conditioning and application assembly (1) comprising:
a container (3) containing a nail varnish composition comprising a cosmetically acceptable medium and at least one thixotropic thickener, the composition being of thixotropic nature,
an applicator (2) for taking up the product contained in the container and applying it to the nails, comprising a flexible blade (6) defining an application face (15).

A subject of the invention is also a conditioning and application assembly (1) comprising:
a container (3) containing a nail varnish composition comprising a cosmetically acceptable medium and at least one thixotropic thickener, the composition having a plateau stiffness modulus Gp of greater than 100 Pa,
an applicator (2) for taking up the product contained in the container and applying it to the nails, comprising a flexible blade (6) defining an application face (15).

A subject of the invention is also a process for making up the nails, which comprises applying to the nails a composition as defined above.

Further subjects of the invention are the use of a composition as defined above for obtaining a homogeneous film of varnish deposited on the nails, which shows good gloss and/or good coverage properties, and the film itself.

The term “cosmetically acceptable medium” means a non-toxic medium that may be applied to human keratin materials, in particular the nails.

The composition according to the invention is of thixotropic nature.

For the purposes of the invention, the term “composition of thixotropic nature”, or thixotropic composition, means a structured composition that fluidizes (in particular its viscosity reduces) when a non-chemical action is applied thereto, in particular a mechanical action, and which recovers all or part of its initial viscosity after a sufficient standing time that may be more or less long, at room temperature.

In particular, the composition has the following properties:
the composition has shear-thinning properties, i.e. the viscosity of the composition decreases when increasing shears are applied to the composition;
after applying an intense shear, the composition becomes fluid (in particular its viscosity decreases). The viscosity, consistency and elasticity of the composition, after destructuring it, in particular after a standing time of one minute after having applied the shear, are lower than those of the composition before applying the intense shear;
the composition partially or totally regenerates its initial structure after a sufficient standing time and the restructuring of the composition is delayed over time. The restructuring of the composition therefore does not take place instantaneously, but rather is delayed over time.

A definition of a thixotropic composition is especially given in the book “Comprendre la rhéologie—de la circulation du sang à la prise du béton” by P. Coussot and J. L. Grossiord, EDP Science, 2002, pages 16 and 17.

The thixotropic behavior of the composition may especially be characterized by the viscosity measurements of the composition at a low shear rate and then at a high shear rate, as described below:

These measurements are taken on a Haake RheoStress® RS 600 controlled-stress rheometer from the company ThermoRheo, equipped with a thermostatically controlled bath and a stainless steel spindle with cone/plate geometry, of diameter 35 mm and angle 2°, with a gap of 0.104 mm. The two surfaces are “sanded” to limit the slipping on the walls. An anti-evaporation device (solvent bell-jar) is used.

The measurements are taken at 20° C.±1° C.

In a first stage, the sample is placed at a temperature of 20° C.±1° C. for 300 seconds (without any applied shear).
a) increasing shears are applied to the sample, starting from an initial stress of 0 Pa to arrive at a final stress of 500 Pa, the stresses being applied once only (which corresponds to a shear gradient ranging for example from 10⁻¹ s⁻¹ to 400 s⁻¹). The change in the viscosity η as a function of the shear gradient \dot{γ} is then plotted. The measurement is taken on 40 logarithmically distributed points. A stable value between each stress is awaited, the waiting time between each stress being 30 s.
b) The composition is then destructured by applying thereto a continuous shear corresponding to a stress of 500 Pa (corresponding to the shear of 400 s⁻¹) for 30 seconds.
c) Increasing stresses are applied to the sample, starting from an initial stress of 500 Pa to arrive at a final stress of 0 Pa, the stresses being applied once only.
   The measurement is taken on 40 logarithmically distributed points.
   A stable value between each stress is awaited, the waiting time between each stress being 30 s.
   The shear gradient range is from 400 s⁻¹ to 10⁻³ s⁻¹. In the range under consideration, the maximum value of 400 s⁻¹ should be taken into account with an uncertainty of measurement of ±10 s⁻¹.
The change in the viscosity η as a function of the shear gradient \dot{γ} is then plotted.
d) The sample is then subjected to a controlled stress of 10 Pa for 1000 s, and the change in viscosity as a function of time is measured.
e) Increasing stresses are again applied to the sample, starting from an initial stress of 0 Pa to arrive at a final stress of 500 Pa, the stresses being applied once only (which corresponds to a shear gradient ranging for example from 10⁻² s⁻¹ to 300 s⁻¹).

The results are analysed by means of a graphical representation of the change in viscosity, noted as η, as a function of the shear gradient, noted as \dot{γ}. The viscosity η, the shear gradient \dot{γ} and the controlled stress τ are related by the relationship \dot{γ}=τ/η

The composition is such that it has a viscosity, as measured during step e), at a shear of 4×10⁻² s⁻¹, ranging for example from 102 to 10⁴ Pa·s, better still from 5×10² to 5×10³ Pa·s and better still from 600 to 4000 Pa·s.

The shear-thinning nature of the composition preferably has a viscosity difference (viscosity measured in step e), at a shear rate of 100 s⁻¹−viscosity measured in step e), at a shear rate of 4×10⁻² s⁻¹) ranging for example from 10 to 10⁵ Pa·s and better still from 10² to 10⁴ Pa·s.

The thixotropic behavior of the composition is characterized by a difference in viscosity measured at a shear rate of 1 s⁻¹ between step c) and step e) (viscosity measured during step c) at a shear rate of 1 s⁻¹−viscosity measured during step e) of at least 1 Pa·s, preferably of at least 10 Pa·s, better still at least 20 Pa·s, even better still of at least 30 Pa·s and preferentially of at least 40 Pa·s.

In particular, the difference in viscosity measured at a shear rate of 1 s⁻¹ between step c) and step e) is the difference (viscosity measured during step c) at a shear rate of 1 s⁻¹−viscosity measured during step e) ranging for example from 1 to 1000 Pa·s, better still from 20 to 500 Pa·s and in particular from 40 to 200 Pa·s.

### Measurement of the Viscoelastic Properties

The compositions in accordance with the invention advantageously have viscoelastic behavior, with a dominant elastic nature.

In general, a material is said to be viscoelastic when, under the effect of shear, it has both the characteristics of an elastic material, i.e. capable of storing energy, and the characteristics of a viscous material, i.e. capable of dissipating energy.

More particularly, the viscoelastic behavior of the compositions in accordance with the invention may be characterized by its stiffness modulus G*, its elasticity δ and its flow threshold τ_{c}; these parameters are defined especially in the publication “Initiation à la rhéologie [Introduction to Rheology]”, G. Couarraze and J. L. Grossiord, 2nd edition, 1991, published by Lavoisier-Tec 1 Doc.

The measurements are taken using a Haake RheoStress 600® controlled-stress rheometer from the company ThermoRheo, equipped with a thermostatically controlled bath and a stainless-steel spindle with plate/plate geometry, the plate having a diameter of 20 mm and a gap (distance between the lower plate—known as the stator plate—on which the composition is deposited, and the upper plate—known as the rotor plate) of 1 mm. The two plates are striated to limit the sliding at the walls of the plates. An anti-evaporation device (solvent bell-jar) is used.

The measurements are taken at 20° C.±1° C.

The composition is subjected to a harmonic shear according to a stress τ(t) varying sinusoidally according to a pulse ω (ω=2Πν), ν being the frequency of the applied shear.

The stress τ(t) and the strain γ(t) are defined, respectively, by the following relationships:

τ(t)=τ₀ cos(ω·t) γ(t)=γ₀ cos(ω·t−δ)

τ₀ being the maximum amplitude of the stress and γ₀ being the maximum amplitude of the strain. δ is the dephasing angle between the stress and the strain and G* corresponds to the ratio τ₀ to γ₀.

The measurements are performed at a set frequency of 1 Hz (ν=1 Hz).

In a first stage, the sample is placed at a temperature of 20° C.±1° C. for 300 seconds.

Increasing stresses are then applied to the sample, starting from an initial stress equal to 0.01 Pa to arrive at a final stress of 1000 Pa, up to the point of destruction of the sample (the stresses being applied once only).

The change in the stiffness modulus G* (corresponding to the ratio of τ₀ to γ₀) and the dephasing δ (corresponding to the dephasing angle of the applied stress relative to the measured deformation) are measured as a function of the applied stress τ(t).

In particular, the deformation of the composition for the stress region in which the variation of the stiffness modulus G* and of the elasticity δ is less than 7% (microdeformation region) is measured, and the “plateau” parameters Gp and δₚ are thus determined.

The threshold stress τ_{c} (since the composition does not flow under its own weight, the threshold stress corresponds to the minimum force that it is necessary to apply to the composition to cause it to flow) is determined from the curve G*=f(τ). It is defined as the value of τ at the intersection of the two tangents to the curve G*=f(τ) for low values of τ and for high values of τ.

The viscoelastic behavior of the compositions according to the invention is especially characterized by a plateau stiffness modulus Gp of greater than 100 Pa and preferably greater than 500 Pa.

Accordingly, a subject of the invention is also a nail varnish composition comprising a cosmetically acceptable medium and at least one thixotropic thickener, the composition preferably having a plateau stiffness modulus Gp of greater than 100 Pa.

In particular, the compositions according to the invention have a plateau stiffness modulus Gp ranging for example from 100 to 2×10⁶ Pa·s, preferably from 5×10² to 10⁴ Pa·s, better still from 800 to 4000 Pa·s and in particular from 1000 to 3000 Pa·s.

The compositions in accordance with the invention may moreover preferably have an elasticity δₚ ranging for example from 20 to 300 and in particular ranging for example from 150 to 250 and a flow threshold τ_{c} ranging for example from 10 Pa to 3×10⁴ Pa, in particular ranging for example from 30 Pa to 500 Pa and better still from 50 to 200 Pa.

### Thickener

The composition according to the invention comprises a thixotropic thickener in an amount that is sufficient to give the composition a viscosity at rest sufficient to give it its texture, and thixotropic behavior.

In particular, the nature and/or amount of the thickener is (are) such that, in response to a non-chemical action, especially a mechanical action, prior to or simultaneously with the application of the composition to the nails, the viscosity of the composition may be reversibly lowered to a value not exceeding 0.4 Pa·s and preferably to a value not exceeding 0.3 Pa·s.

The thixotropic thickener may for example be present in a content of greater than or equal to 1.7% by weight, for example ranging for example from 1.7% to 15% by weight, preferably greater than or equal to 2% by weight, for example ranging for example from 2% to 10% by weight and preferentially ranging for example from 2% to 7.5% by weight, preferably ranging for example from 3% to 7.5% by weight, preferably ranging for example from 3.5% to 7.5% by weight, relative to the total weight of the composition.

The thickener may be chosen from hydrophilic or organophilic clays, hydrophilic or hydrophobic fumed silicas, and elastomeric organopolysiloxanes, and mixtures thereof.

Clays are silicates containing a cation that may be chosen from calcium, magnesium, aluminium, sodium, potassium and lithium cations, and mixtures thereof. The term “hydrophilic clay” means a clay that is capable of swelling in water; this clay swells in water and forms after hydration a colloidal dispersion.

Examples of such products that may be mentioned include clays of the smectite family such as montmorillonites, hectorites, bentonites, beidellites and saponites, and also of the family of vermiculites, stevensite and chlorites.

These clays may be of natural or synthetic origin.

Hydrophilic clays that may be mentioned include smectites such as saponites, hectorites, montmorillonites, bentonites or beidellite and in particular synthetic hectorites (also known as laponites), for instance the products sold by the company Laporte under the names Laponite XLG, Laponite RD and Laponite RDS (these products are sodium magnesium silicates and in particular sodium lithium magnesium silicates); bentonites, for instance the product sold under the name Bentone HC by the company Rheox; magnesium aluminium silicates, which are especially hydrated, for instance the products sold by the company Vanderbilt Company under the names Veegum Ultra, Veegum HS and Veegum DGT, or alternatively calcium silicates and especially the product in synthetic form sold by the company under the name Micro-cel C.

The organophilic clays are clays modified with chemical compounds that make the clay capable of swelling in solvent media.

The clay may be chosen from montmorillonite, bentonite, hectorite, attapulgite and sepiolite, and mixtures thereof. The clay is preferably a bentonite or a hectorite.

The organophilic clays are clays modified with a chemical compound chosen from quaternary amines, tertiary amines, amine acetates, imidazolines, amine soaps, fatty sulfates, alkyl aryl sulfonates and amine oxides, and mixtures thereof.

Organophilic clays that may be mentioned include quaternium-18 bentonites such as those sold under the names Bentone 3, Bentone 38 and Bentone 38V by the company Elementis, Tixogel VP by the company United Catalyst, and Claytone 34, Claytone 40 and Claytone XL by the company Southern Clay; stearalkonium bentonites such as those sold under the names Bentone 27V by the company Elementis, Tixogel LG by the company United Catalyst, and Claytone AF and Claytone APA by the company Southern Clay; quaternium-18/benzalkonium bentonites such as those sold under the names Claytone HT and Claytone PS by the company Southern Clay.

The hydrophilic fumed silicas may be obtained by high-temperature hydrolysis of a volatile silicon compound in an oxyhydric flame, producing a finely divided silica. Hydrophilic silicas have a large number of silanol groups at their surface. Such hydrophilic silicas are sold, for example, under the names Aerosil 130®, Aerosil 200®, Aerosil 255®, Aerosil 300® and Aerosil 380® by the company Degussa, Cab-O-Sil HS-5®, Cab-O-Sil EH-5®, Cab-O-Sil LM-130®, Cab-O-Sil MS-55® and Cab-O-Sil M-5® by the company Cabot.

The hydrophobic fumed silicas may be obtained by modification of the surface of the silica via a chemical reaction that generates a reduction in the number of silanol groups, these groups possibly being substituted especially with hydrophobic groups.

The hydrophobic groups may be:
trimethylsiloxyl groups, which are especially obtained by treating fumed silica in the presence of hexamethyldisilazane. Silicas thus treated are known as “Silica silylate” according to the CTFA (6th edition, 1995). They are sold, for example, under the references Aerosil R812® by the company Degussa, and Cab-O-Sil TS-530® by the company Cabot,
dimethylsilyloxyl or polydimethylsiloxane groups, which are especially obtained by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The elastomeric polyorganosiloxanes are generally partially or totally crosslinked and possibly of three-dimensional structure.

The elastomeric polyorganosiloxanes combined with a fatty phase are generally in the form of a gel consisting of an elastomeric polyorganosiloxane combined with a fatty phase, included in at least one hydrocarbon-based oil and/or at least one silicone oil. They may be chosen especially from the crosslinked polymers described in patent application EP-A-0 295 886.

According to the patent application, the elastomeric organopolysiloxanes are obtained by addition reaction and crosslinking of at least:

(a) one organopolysiloxane containing at least two lower alkenyl groups per molecule;
(b) one organopolysiloxane containing at least two hydrogen atoms linked to a silicon atom per molecule; and
(c) a platinum-type catalyst.

The elastomeric organopolysiloxanes combined with a fatty phase may also be chosen from those described in U.S. Pat. No. 5,266,321, especially from polyorganopoly-siloxanes comprising units R₂SiO and RSiO_{1.5} and possibly units R₃SiO_{0.5} and/or SiO₂ in which the radicals R, independently of each other, denote a hydrogen, an alkyl such as methyl, ethyl or propyl, an aryl such as phenyl or tolyl, or an unsaturated aliphatic group such as vinyl, and in which the weight ratio of the units R₂SiO to the units RSiO_{1.5} ranges from 1/1 to 30/1.

According to one preferred embodiment, the thixotropic thickener is chosen from organophilic modified clays such as hectorite modified with benzyldimethylammonium stearate.

According to one advantageous embodiment, the thixotropic thickener comprises, besides an organophilic modified clay, a hydrophilic fumed silica.

### Additional Thickener

The composition according to the invention may also comprise an additional thickener different from and in addition to the thixotropic thickeners described previously.

This additional thickener is not capable by itself of giving the composition the thixotropic nature (non-thixotropic thickener); it especially makes it possible to adjust the viscosity of the composition to obtain uniform flow.

The additional thickener may be chosen, according to the cosmetically acceptable medium of the composition, from:
hydrophilic thickeners such as guar gum, quaternized guar gum, nonionic guar gums, xanthan gum, carob gum, scleroglucan gum, gellan gum, rhamsan gum, karaya gum, alginates, maltodextrin, starch and derivatives thereof, and hyaluronic acid and salts thereof,
polyglyceryl(meth)acrylate polymers,
polyvinylpyrrolidone,
polyvinyl alcohol,
crosslinked acrylamide polymers and copolymers,
associative polymers and especially associative polyurethanes,
organophilic thickeners, for instance:
alkyl guar gums (with a C₁-C₆ alkyl group), such as those described in EP-A-708 114;
oil-gelling polymers, for instance triblock or star polymers resulting from the polymerization or copolymerization of at least one monomer containing an ethylenic group, for instance the polymers sold under the name Kraton;
polyamides resins comprising alkyl groups containing from 12 to 22 carbon atoms, such as those described in U.S. Pat. No. 5,783,657;
hydrophobic thickeners such as polysaccharide alkyl ethers (especially in which the alkyl group contains from 1 to 24, preferably from 1 to 10, better still from 1 to 6 and more especially from 1 to 3 carbon atoms), such as those described in document EP-A-898 958.

The additional thickener may for example be present in a content ranging for example from 0.1% to 20% by weight and preferably ranging for example from 0.1% to 10% by weight relative to the total weight of the composition.

### Organic Solvent Medium

The composition according to the invention may comprise an organic solvent medium comprising at least one organic solvent chosen from:
ketones that are liquid at room temperature, such as methyl ethyl ketone, methyl isobutyl ketone, diisobutyl ketone, isophorone, cyclohexanone or acetone;
alcohols that are liquid at room temperature, such as ethanol, isopropanol, diacetone alcohol, 2-butoxyethanol or cyclohexanol;
propylene glycol ethers that are liquid at room temperature, such as propylene glycol monoethyl ether, propylene glycol monoethyl ether acetate or dipropylene glycol n-butyl ether;
cyclic ethers such as γ-butyrolactone;
short-chain esters (containing from 3 to 8 carbon atoms in total), such as ethyl acetate, butyl acetate, methyl acetate, propyl acetate, isopropyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane and cyclohexane;
alkyl sulfoxides such as dimethyl sulfoxide;
aldehydes that are liquid at room temperature, such as benzaldehyde or acetaldehyde;
ethyl 3-ethoxypropionate;
carbonates such as propylene carbonate or dimethyl carbonate;
acetals such as methylal;
and mixtures thereof.

The organic solvent medium may represent from 30% to 97% by weight and especially from 50% to 95% by weight relative to the total weight of the composition.

The composition according to the invention may comprise an aqueous medium.

The aqueous medium content of the composition may for example range from 5% to 95% by weight and preferably from 50% to 70% by weight relative to the total weight of the composition.

### Film-Forming Polymer

The composition advantageously comprises at least one film-forming polymer.

According to the present invention, the term “film-forming polymer” means a polymer that is capable, by itself or in the presence of an auxiliary film-forming agent, of forming a continuous film that adheres to a support and especially to keratin materials.

Among the film-forming polymers that may be used in the composition of the present invention, mention may be made of synthetic polymers, of free-radical type or of polycondensate type, and polymers of natural origin, and mixtures thereof.

The film-forming polymer may be chosen in particular from cellulose-based polymers such as nitrocellulose, cellulose acetate, cellulose acetobutyrate, cellulose acetopropionate and ethylcellulose, or alternatively polyurethanes, acrylic polymers, vinyl polymers, polyvinyl butyrals, alkyd resins, resins derived from aldehyde condensation products such as arylsulfonamide-formaldehyde resins, for instance toluenesulfonamide-formaldehyde resin, arylsulfonamide-epoxy resins or ethyltosylamide resins.

Film-forming polymers that may especially be used include nitrocellulose RS ⅛ sec.; RS ¼ sec.; RS ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; SS 5 sec.; sold especially by the company Hercules; the toluenesulfonamide-formaldehyde resins Ketjenflex MS80 from the company Akzo, Santolite MHP or Santolite MS 80 from the company Faconnier, or Resimpol 80 from the company Pan Americana, the alkyd resin Beckosol ODE 230-70-E from the company Dainippon, the acrylic resin Acryloid B66 from the company Rohm & Haas, and the polyurethane resin Trixene PR 4127 from the company Baxenden.

According to one embodiment of the invention, the film-forming polymer is a film-forming linear block ethylenic polymer, which preferably comprises at least one first block and at least one second block with different glass transition temperatures (Tg), the first and second blocks being linked together by an intermediate block comprising at least one constituent monomer of the first block and at least one constituent monomer of the second block.

Advantageously, the first and second blocks of the block polymer are mutually incompatible.

Such polymers are described, for example, in documents EP 1 411 069 or WO 04/028 488.

The film-forming polymer may be present in the composition according to the invention in a dry matter content ranging for example from 0.1% to 60% by weight, preferably ranging for example from 2% to 40% by weight and better still from 5% to 25% by weight relative to the total weight of the composition.

### Auxiliary Film-Forming Agent

To improve the film-forming properties of the nail varnish composition, an auxiliary film-forming agent may be provided.

Such an auxiliary film-forming agent may be chosen from any compound known to those skilled in the art as being capable of satisfying the desired function, and may be chosen especially from plasticizers and coalescers for the film-forming polymer(s).

Thus, the composition may also comprise at least one plasticizer and/or one coalescer. In particular, mention may be made, alone or as a mixture, of common plasticizers and coalescers, such as:
glycols and derivatives thereof, such as diethylene glycol ethyl ether, diethylene glycol methyl ether, diethylene glycol butyl ether or diethylene glycol hexyl ether, and ethylene glycol ethyl ether, ethylene glycol butyl ether or ethylene glycol hexyl ether;
glycol esters;
propylene glycol derivatives and in particular propylene glycol phenyl ether, propylene glycol diacetate, dipropylene glycol ethyl ether, tripropylene glycol methyl ether, diethylene glycol methyl ether and propylene glycol butyl ether;
acid esters and especially carboxylic acid esters, such as citrates, phthalates, adipates, carbonates, tartrates, phosphates and sebacates;
oxyethylenated derivatives, such as oxyethylenated oils, especially plant oils such as castor oil; and
mixtures thereof.

The type and amount of plasticizer and/or coalescer may be chosen by a person skilled in the art on the basis of his general knowledge.

For example, the plasticizer and/or coalescer content may range for example from 0.01% to 20% and in particular from 0.5% to 10% by weight relative to the total weight of the composition.

### Spreading Agent

According to one embodiment, the composition according to the invention comprises a spreading agent intended to promote the application of the composition to the nails. It may be chosen from linear or cyclic silicone oils, especially those with a viscosity≦6 centistokes (6×10⁻⁶ m²/s) and especially containing from 3 to 6 silicon atoms, these silicones optionally comprising one or more alkyl or alkoxy groups containing 1 or 2 carbon atoms. As silicone oils that may be used in the invention, mention may be made especially of octa-methylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The spreading agent may for example represent from 0.1% to 15% by weight, preferably from 0.5% to 10% by weight and better still from 1% to 5% by weight relative to the total weight of the composition.

### Coloring Agent

According to one alternative, the coloring agent is chosen from magnetic substances, diffractive pigments, interference pigments and reflective particles, and mixtures thereof.

The coloring agent may for example be present in a content of greater than or equal to 2.5% by weight, preferably greater than or equal to 3% by weight, better still greater than or equal to 3.5% by weight and even better still greater than or equal to 4% by weight, which may be up to 20%, preferably up to 15% by weight and better still up to 10% by weight relative to the total weight of the composition.

### A/ Magnetic Particles

The term “magnetic particles”, also referred to as magnetic substances, denotes particles with magnetic susceptibility, i.e. which are sensitive to the action of a magnetic field and tend, for example, to align with the field lines.

The magnetic particles may comprise any magnetic material that is sensitive to the lines of a magnetic field, whether it is produced by a permanent magnet or derived from an induction, this material being chosen, for example, from nickel, cobalt, iron, alloys and oxides thereof, especially Fe₃O₄, and also gadolinium, terbium, dysprosium, erbium, and alloys and oxides thereof. The magnetic material may be of “soft” or “hard” type.

The magnetic particles may or may not have a multilayer structure, comprising at least one layer of a magnetic material, for instance iron, nickel, cobalt, or alloys and oxides thereof, especially Fe₃O₄.

The magnetic particles are preferably of anisotropic nature. The magnetic particles are preferably aspherical, for example having an elongated shape. Thus, when these particles are subjected to a magnetic field, they tend to become oriented with their longitudinal axis in the alignment of the field lines, and undergo a change of orientation that is reflected by a change in appearance arising from the anisotropy and creating the pattern(s).

When the magnetic particles are substantially spherical, their appearance is preferably non-uniform, such that a change in orientation induces a change in appearance.

The composition may take a form that prevents any new change in orientation of the magnetic particles under the effect of a magnetic field after a given drying time.

The size of the magnetic particles is, for example, between 1 nm and 700 μm, for example between 1 μm and 500 μm and better still between about 10 μm and about 150 μm. The term “size” denotes the dimension given by the statistical granulometric distribution to half the population, noted as D50.

The magnetic particles may comprise magnetic pigments.

Pigments that are most particularly suitable for use are nacres comprising iron oxide Fe₃O₄. Pigments with magnetic properties are, for example, those sold under the trade names Colorona Blackstar Blue, Colorona Blackstar Green, Colorona Blackstar Gold, Colorona Blackstar Red, Microna Matte Black (17437), Mica Black (17260), Colorona Patina Silver (17289) and Colorona Patina Gold (17288) from the company Merck, and Gemtone Moonstone (G 004) or Chromalite Black (4498) from the company Engelhard.

As examples of magnetic pigments that may be included in the formulation of the composition, mention may be made of black iron oxide particles, for example those sold under the name Sicovit Black E172 by the company BASF.

The magnetic pigments may also comprise iron metal, especially passivated soft iron, for example obtained from iron carbonyl using the process described in U.S. Pat. No. 6,589,331, the content of which is incorporated by reference. These particles may comprise a surface layer of an oxide.

### B/ Interference Pigment

The term “interference pigment” denotes a pigment capable of producing a color via an interference phenomenon, for example between the light reflected by a plurality of superposed layers of different refractive indices, especially a succession of layers with high and low refractive indices.

An interference pigment may comprise, for example, more than four layers with different refractive indices.

The layers of the interference pigment may or may not surround a core, which may or may not have a flattened shape.

Nacres are examples of interference pigments.

### Nacres

The term “nacre” should be understood as meaning colored particles of any form, which may or may not be iridescent, especially produced by certain molluscs in their shell, or alternatively synthesized, and which have a color effect via optical interference.

Examples of nacres that may be mentioned include nacreous pigments such as titanium mica coated with an iron oxide, mica coated with bismuth oxychloride, titanium mica coated with chromium oxide, titanium mica coated with an organic dye especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or tint.

As illustrations of nacres that may be introduced as interference pigments into the first composition, mention may be made especially of the gold-colored nacres sold especially by the company Engelhard under the name Brillant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

### Reflective Interference Particles

These particles may be chosen from particles with a synthetic substrate coated at least partially with at least one layer of at least one metal oxide, chosen, for example, from titanium oxide, especially TiO₂, iron oxide, especially Fe₂O₃, tin oxide, chromium oxide, barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MOS₂, and mixtures or alloys thereof.

As examples of such particles, mention may be made, for example, of particles comprising a synthetic mica substrate coated with titanium dioxide, or glass particles coated with either brown iron oxide, titanium oxide, tin oxide or a mixture thereof, for instance those sold under the brand name Reflecks® by the company Engelhard. The particles with a glass substrate coated with a metal oxide, especially TiO₂, are sold by the company Nippon Sheet Glass under the name Metashine.

### Goniochromatic Pigment

For the purposes of the present invention, the term “goniochromatic pigment” means a coloring agent for obtaining, when the composition is spread onto a support, a color trajectory in the plane a*b* of the CIE 1976 calorimetric space that corresponds to a variation Dh° of the angle of hue h° of at least 20° when the angle of observation is varied relative to the normal between 0° and 80°, for an angle of light incidence of 45°.

The color trajectory may be measured, for example, using an Instrument Systems brand spectrogonio-reflectometer of reference GON 360 Goniometer, after the first composition has been spread in fluid form to a thickness of 300 μm using an automatic spreader onto an Erichsen brand contrast card of reference Typ 24/5, the measurement being taken on the black background of the card.

The goniochromatic pigment may be chosen, for example, from multilayer interference structures and liquid-crystal coloring agents.

In the case of a multilayer structure, it may comprise, for example, at least two layers, each layer being made, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MOS₂, cryolite, alloys and polymers, and combinations thereof. The multilayer structure may or may not have, relative to a central layer, symmetry regarding the chemical nature of the stacked layers. Different effects are obtained depending on the thickness and the nature of the various layers.

Examples of symmetrical multilayer interference structures are, for example, the following structures: Fe₂O₃/SiO₂/Fe₂O₃/SiO₂/Fe₂O₃, a pigment having this structure being sold under the name Sicopearl by the company BASF; MOS₂/SiO₂/mica-oxide/SiO₂/MOS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the name Xirona by the company Merck (Darmstadt).

The liquid-crystal coloring agents comprise, for example, silicones or cellulose ethers on which are grafted mesomorphic groups. Liquid-crystal goniochromatic particles that may be used, for example, are those sold by the company Chemx and also those sold under the name Helicone® HC by the company Wacker.

Goniochromatic pigments that may also be used include certain nacres, pigments with effects on synthetic substrate, especially a substrate of alumina, silica, borosilicate, iron oxide or aluminium type, or interference holographic flakes derived from a polyterephthalate film.

The material may also comprise dispersed goniochromatic fibres. Such fibres may be less than 80 μm long, for example.

### C/ Diffractive Pigment

For the purposes of the present invention, the term “diffractive pigment” denotes a pigment capable of producing a color variation according to the angle of observation when lit with white light, on account of the presence of a structure that diffracts light. Such a pigment is also occasionally known as a holographic pigment.

A diffractive pigment may comprise a diffracting network capable, for example, of diffracting an incident monochromatic light ray in defined directions.

The diffraction network may comprise a periodic unit, especially a line, the distance between two adjacent units being of the same order of magnitude as the wavelength of the incident light.

When the incident light is polychromatic, the diffraction network will separate the various spectral components of the light and produce a rainbow effect.

Reference may appropriately be made regarding the structure of diffractive pigments to the article “Pigments Exhibiting Diffractive Effects” by Alberto Argoitia and Matt Witzman, 2002, Society of Vacuum coaters, 45^{th} Annual Technical Conference Proceedings 2002.

The diffractive pigment may be made with units having different profiles, especially triangular, symmetrical or non-symmetrical, in gaps, of constant or non-constant width, sinusoidal, in ladder form.

The spatial frequency of the network and the depth of the units will be chosen as a function of the degree of separation of the various orders desired. The frequency may range, for example, between 500 and 3000 lines per mm.

Preferably, the particles of the diffractive pigment each have a flattened form, and are especially in the form of platelets.

The same pigment particle may comprise two crossed, perpendicular or non-perpendicular diffraction networks, of identical or different ruling.

The diffractive pigment may have a multilayer structure comprising a layer of a reflective material, covered at least on one side with a layer of a dielectric material. The latter material may give the diffractive pigment better rigidity and durability. The dielectric material may thus be chosen, for example, from the following materials: MgF₂, SiO₂, Al₂O₃, AlF₃, CeF₃, LaF₃, NdF₃, SmF₂, BaF₂, CaF₂, LiF and combinations thereof. The reflective material may be chosen, for example, from metals and alloys thereof, and also from non-metallic reflective materials. Among the metals that may be used, mention may be made of Al, Ag, Cu, Au, Pt, Sn, Ti, Pd, Ni, Co, Rd, Nb, Cr and Fe, and materials, combinations or alloys thereof, and doping products thereof with rare-earth metals.

Such a reflective material may, by itself, constitute the diffractive pigment, which will then be monolayer.

As a variant, the diffractive pigment may comprise a multilayer structure comprising a core of a dielectric material covered with a reflective layer at least on one side, or even totally encapsulating the core. A layer of a dielectric material may also cover the reflective layer(s). The dielectric material used is then preferably mineral, and may be chosen, for example, from metal fluorides, metal oxides, metal sulfides, metal nitrides, and metal carbides, and combinations thereof. The dielectric material may be in crystalline, semi-crystalline or amorphous form. In this configuration, the dielectric material may be chosen, for example, from the following materials: MgF₂, SiO, SiO₂, Al₂O₃, TiO₂, WO, AlN, BN, B₄C, WC, TiC, TiN, N₄Si₃, ZnS, glass particles and carbons of diamond type, and combinations thereof.

As a variant, the diffractive pigment may be composed of a preformed dielectric or ceramic material such as a mineral in natural leaflet form, for example mica perovskite or talc, synthetic leaflets formed from glass, alumina, SiO₂, carbon, an iron oxide/mica, mica coated with BN, BC, graphite or bismuth oxychloride, and combinations thereof.

Instead of a layer of a dielectric material, other materials that improve the mechanical properties may be suitable for use. Such materials may comprise silicone, metal silicides, semiconductive materials formed from elements of groups III, IV and V, metals with a cubic-centered crystal structure, cermet compositions or materials and semiconductive glasses, and various combinations thereof.

The diffractive pigment used may be chosen especially from those described in the American patent application US 2003/0 031 870 published on 13 Feb. 2003.

A diffractive pigment may comprise, for example, the following structure: MgF₂/Al/MgF₂, a diffractive pigment having this structure being sold under the name Spectraflair 1400 Pigment Silver by the company Flex Products, or Spectraflair 1400 Pigment Silver FG. The weight proportion of MgF₂ may be between 80% and 95% of the total weight of the pigment.

Other diffractive pigments are sold under the names Metalure® Prismatic by the company Eckart.

Other possible structures are Fe/Al/Fe or Al/Fe/Al.

The size of the diffractive pigment may be, for example, between 5 and 200 μm and better still between 5 and 100 μm, for example between 5 and 30 μm.

The thickness of the diffractive pigment particles may be less than or equal to 3 μm and better still 2 μm, for example about 1 μm.

### D/ Reflective Particles or Pigments

The term “reflective particles” denotes particles whose size, structure, especially the thickness of the layer(s) of which they are made and of their physical and chemical nature, and surface state allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

The reflective particles may be selected so as not to significantly alter the coloration effect generated by the coloring agents with which they are combined, and more particularly so as to optimize this effect in terms of color yield. They may more particularly have a yellow, pink, red, bronze, orange, brown, gold and/or coppery color or tint.

These particles may have varied forms and may especially be in platelet or globular form, in particular spherical.

Irrespective of their form, the reflective particles may or may not have a multilayer structure, and, in the case of a multilayer structure, for example at least one layer of uniform thickness, especially a reflective material.

When the reflective particles do not have a multilayer structure, they may be composed, for example, of metal oxides, especially titanium or iron oxides obtained synthetically.

When the reflective particles have a multilayer structure, they may comprise, for example, a natural or synthetic substrate, especially a synthetic substrate at least partially coated with at least one layer of a reflective material, especially of at least one metal or metallic material. The substrate may be a monomaterial, multimaterial, organic and/or mineral substrate.

More particularly, it may be chosen from glasses, ceramics, graphite, metal oxides, aluminas, silicas, silicates, especially aluminosilicates and borosilicates, and synthetic mica, and mixtures thereof, this list not being limiting.

The reflective material may comprise a layer of metal or of a metallic material.

Reflective particles are described especially in documents JP-A-09188830, JP-A-10158450, JP-A-10158541, JP-A-07258460 and JP-A-05017710.

Again as an example of reflective particles comprising a mineral substrate coated with a layer of metal, mention may also be made of particles comprising a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the name Crystal Star GF 550 and GF 2525 by this same company.

Particles comprising a metallic substrate such as silver, aluminium, iron, chromium, nickel, molybdenum, gold, copper, zinc, tin, manganese, steel, bronze or titanium, may also be used, the substrate being coated with at least one layer of at least one metal oxide such as titanium oxide, aluminium oxide, iron oxide, cerium oxide, chromium oxide or silicon oxides, and mixtures thereof.

Examples that may be mentioned include aluminium powder, bronze powder or copper powder coated with SiO₂ sold under the name Visionaire by the company Eckart.

### Additional Dyestuff

According to the alternative described above, the composition according to the invention may comprise, besides the coloring agents chosen from magnetic substances, diffractive pigments, interference pigments and reflective particles, at least one “additional” coloring agent, which especially produces a color by absorption of at least part of the visible spectrum.

These additional dyestuffs may be chosen from water-soluble or liposoluble dyes, and organic or inorganic or hybrid pigments.

The coloring agent may be a particulate or non-particulate compound.

The liposoluble dyes are, for example, Sudan red, DC Red 17, DC Green 6, β-carotene, soybean oil, Sudan brown, DC Yellow 11, DC Violet 2, DC Orange 5 and quinoline yellow.

The water-soluble dyes are, for example, beetroot juice and methylene blue.

The coloring agent may also be an organic pigment or lake chosen from the materials below, and mixtures thereof:
cochineal carmine,
organic pigments of azo, anthraquinone, indigoid, xanthene, pyrene, quinoline, triphenylmethane or fluorane dyes, in particular pigments of D&C type,
organic lakes or insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

The chemical materials corresponding to each of the organic dyestuffs mentioned above are mentioned in the publication “International Cosmetic Ingredient Dictionary and Handbook”, 1997 edition, pages 371 to 386 and 524 to 528, published by “The Cosmetic, Toiletry, and Fragrance Association”, the content of which is incorporated into the present patent application by reference.

The additional coloring agents may be present for example in a content ranging for example from 0.01% to 30% by weight and preferably from 1% to 10% by weight relative to the weight of the composition.

According to another alternative, the composition according to the invention may also comprise one or more dyestuffs chosen from coloring agents and the additional dyestuff described above. In this case, the coloring agents may be present in the composition in a content ranging for example from 0.01% to 60% by weight and preferably from 1% to 40% by weight relative to the weight of the composition.

The composition according to the invention may also comprise one or more fillers, especially in a content ranging for example from 0.01% to 50% by weight and preferably ranging for example from 0.01% to 30% by weight relative to the total weight of the composition. The term “fillers” should be understood as meaning colorless or white, mineral or synthetic particles of any form, which are insoluble in the medium of the composition irrespective of the temperature at which the composition is manufactured. These fillers serve especially to modify the rheology or the texture of the composition.

The fillers may be mineral or organic and of any form, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powder (Orgasol® from Atochem), poly-β-alanine powder and polyethylene powder, tetrafluoroethylene polymer (Teflon®) powder, lauroyllysine, starch, boron nitride, hollow polymer microspheres such as polyvinylidene chloride/acrylonitrile microspheres, for instance Expancel® (Nobel Industrie), acrylic acid copolymer microspheres (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxy-apatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

### Other Additives

The composition may also comprise other ingredients commonly used in cosmetic compositions. Such ingredients may be chosen from spreading agents, wetting agents, dispersants, antifoams, preserving agents, UV-screening agents, active agents, surfactants, moisturizers, fragrances, neutralizers, stabilizers and antioxidants.

Needless to say, a person skilled in the art will take care to select this or these optional additional compound(s), and/or the amount thereof, such that the advantageous properties of the composition for the use according to the invention are not, or are not substantially, adversely affected by the envisaged addition.

### Applicator

The applicator used according to a preferred example of the present invention comprises a flexible blade defining an application face.

Preferably, the flexible blade is incurved so as to define a generally concave application face and may have a convex distal edge, when viewed from above.

By way of example, according to one embodiment, the applicator comprises an incurved flexible blade having an application face defining a developable, for example cylindrical, surface.

According to another embodiment, the applicator comprises an incurved flexible blade defining a generally concave application face and having a convex distal edge when viewed from above.

According to one embodiment, the container containing the nail varnish is configured to allow the product to be taken up without passing through a draining member.

The features discussed above can be provided in an applicator as a separate component, and also in an assembly comprising an applicator and a container.

The invention can allow the user to measure out relatively precisely the amount of product taken up by the applicator, taking into account the consistency of the product and/or the way in which the product is taken up.

The incurved shape of the flexible blade may especially facilitate the uptake of the product.

Furthermore, the absence of a bundle of bristles makes it possible to avoid the formation of striations during deposition of the product onto the nail.

Finally, the shape of the blade may facilitate application to the nails and may allow a high-quality makeup result to be achieved quickly.

In one implementation example of the invention, the flexible blade has an entirely smooth application face.

The flexible blade may comprise reliefs on the longitudinal sides of the application face, for example ribs. By bearing on the finger and/or on the sides of the nail, these reliefs may allow at least one portion of the application face to be spaced from the nail by a predefined distance, which may facilitate the formation of a deposit of product of controlled thickness.

In one implementation example of the invention, the flexible blade defines opposite bearing surfaces for the fingers, so as to allow the user to modify at will the curvature of the application face by gripping the flexible blade more or less tightly between his or her fingers. These bearing surfaces may be defined, for example, by wings extending on either side of an upper wall of the blade, which defines the back thereof.

The flexible blade may be of non-uniform thickness, in order, for example, to allow increased flexibility during use. This can make it possible to reduce the pressure exerted by the blade on the nail and to improve the comfort when applying the makeup and the quality of the makeup result.

When the flexible blade comprises an upper wall, defining the back thereof with wings located on either side of this upper wall, the upper wall may have a smaller thickness of material than in the region of the wings. During application, the upper wall can incurve more easily. In one variant, the walls are thicker than the back. This may make it possible to more easily deform the distal edge of the blade by pressing on the wings, thus better transmitting the exerted effect. The user can more easily control the deformation of the distal edge. This may also make it possible to further distance the finger-pressing region from that bearing the product.

The flexible blade may be connected to a handle shaft, for example via a thicker portion that may be solid and may have a circular or other cross section. The flexible blade may be made as a single component with the handle shaft.

The flexible blade may be made of a thermoplastic elastomer or of any other material that can give the handle shaft a certain amount of flexibility and compatibility with any solvent(s) contained in the product.

The flexible blade may be made of an elastically deformable material having a hardness of, for example, 25 to 60 Shore A.

The flexible blade may optionally be made of a flexible metal or of a complex comprising several layers of different materials, laminated together or combined by overinjection.

In one implementation example, the flexibility of the blade may be such that the blade flattens out substantially when pressed against a flat surface.

The blade may comprise in at least certain portions armature for reinforcing it and/or a fibre filling.

As mentioned hereinabove, the flexible blade advantageously has a convex distal edge, when viewed from above. The distal edge has, for example, at its apex, a radius of curvature of between 4 and 20 mm.

The flexible blade may have a symmetrical general shape relative to a longitudinal midplane.

The flexible blade may be flocked, where appropriate.

The applicator may comprise a handle shaft to which the blade is attached. As an alternate example, the flexible blade may be made with a handle part obtained from moulding and/or cutting therewith. The longitudinal axis of blade may merge with the longitudinal axis of the handle shaft or of the handle part, or may make a non-zero angle therewith.

When a handle shaft is used, the flexible blade may be, for example, immovably attached to the handle shaft, in a removable or non-removable manner.

The applicator may comprise, where appropriate, a member for controlling the curvature of the application face, which may or may not be configured to serve as a handle member.

Thus, the flexible blade may be adjustably attached to the abovementioned handle shaft. The handle shaft and the flexible blade may interact so as to allow the user to vary the shape of the application face, in order, for example, to adapt it to the shape of the treated nail and/or to the consistency of the product.

Also by way of example, the applicator comprises a handle shaft and when the blade is mobile relative to the handle shaft, this handle shaft may be arranged so as to constrain the blade to a greater or lesser extent depending on its position on the handle shaft.

At least one from among the blade and the handle shaft may comprise reliefs allowing an incremental adjustment of the curvature of the application face. As a variant, the adjustment may be performed continuously.

The handle shaft may optionally comprise a mobile control member, the movement of which is accompanied by a change in the curvature.

The change in the curvature of the application face may result from a constraint exerted laterally on two outer opposite sides of the blade, for example, and/or from more or less substantial stretching thereof.

The stress may be exerted on the blade by the handle shaft or by another control member, for example a ring engaged on the blade and axially mobile relative thereto. By moving the ring on the blade, the blade may be more or less tight and may incurve correspondingly.

The blade may be openworked. This may increase the visibility of the nail during the application of makeup and/or may allow the possibility of mounting of the blade on a handle shaft so as to quite simply modify the curvature of the application face. The blade may be openworked, comprising a blade between two opposite edges of the aperture.

The blade may be made, where appropriate, of a transparent material, which may improve the visibility of the nail during use.

Advantageously, the blade is preferably made of a material that facilitates its cleaning, for example a polymer to which the product adheres little, for example a silicone polymer.

According to an example, the applicator may comprise two application faces of different dimensions, these application faces being defined, for example, by two flexible blades arranged end to end. The user may then select the blade that he or she wishes to use for the application, and use the other blade as a handle part. The two blades may be made as a single component by moulding of material or cutting of a sheet, followed by optional shaping.

The applicator may or may not be for single use.

The assembly may be proposed to the user with several applicators corresponding, for example, to the expected number of uses, taking into account the amount of product contained in the container.

The applicators may be separated from each other or provided for use with at least two applicators solidly attached to the same support. This may, where appropriate, facilitate the manufacture and conditioning, the applicators being, for example, moulded and/or precut with the support and separated from the support by the user as and when used.

Several applicators may, for example, be precut on a sheet provided to the user with a container containing the product, for example in a common packaging.

When several applicators are solidly fastened to the same support, the applicators may be identical and may correspond, for example, to several successive uses, or may be different and intended, for example, for the nails of the feet and those of the hands, respectively.

The container may comprise a lid defining a housing for at least partially receiving at least one applicator when not in use.

This housing may optionally extend along an oblique axis relative to the axis of the container, so as to increase the stability of the assembly.

According to another of its aspects, a subject of the invention is also a process for making up the nails, comprising the following steps:
taking up the product in a container using the blade of an applicator as defined above,
applying the product using the application face of the blade.

In such a process, before or after taking up the product, the user can adapt the curvature of the blade to that of the nail either by directly exerting an action on the blade, or by acting on a curvature control member.

According to one embodiment, by way of example, the applicator comprises a flexible blade defining a concave application face, the blade being provided on its longitudinal sides with reliefs for bearing against the nail and/or the finger during application, so as to allow the application face to make with the nail a gap of predefined thickness. The reliefs are, for example, ribs extending on the longitudinal sides of the application face. The reliefs may contribute towards containing the product on the application face, where appropriate.

According to another example, the applicator comprises a flexible blade that has an upper wall and two wings arranged on either side of this upper wall, with the upper wall having a thickness of material different from that of the wings, especially a lower thickness.

The applicator may comprise a flexible blade and a handle shaft to which is adjustably attached the blade, with the blade and the handle shaft cooperating so as to allow, as a function of the position of the blade on the handle shaft, the curvature of the application face to be modified.

According to one embodiment, the applicator may, for example, comprise a flexible blade defining an application face and a member for controlling the curvature of the application face.

The flexible blade may be made as a single component with a handle part. The flexible blade is made, for example, by cutting a material as sheets or by moulding of material with the handle part. The handle part may optionally define an application face.

The flexible blade may be openworked, the application face at least partially extending on either side of this openwork.

The container may comprise a closing blade or a support housed in the container, provided with a housing in which may be at least partially received the flexible blade when not in use.

This housing is defined, for example, by a chimney into which the flexible blade may be inserted when not in use, the flexible blade being, for example, solidly attached to a handle shaft that extends outside the housing. The housing may also be defined by the interior of a closing capsule, which is itself provided with a lid. The support may be arranged to bear on the top end of the container. The closing member may bear on the support at its periphery, so as to contribute towards the leaktightness of the closure, for example.

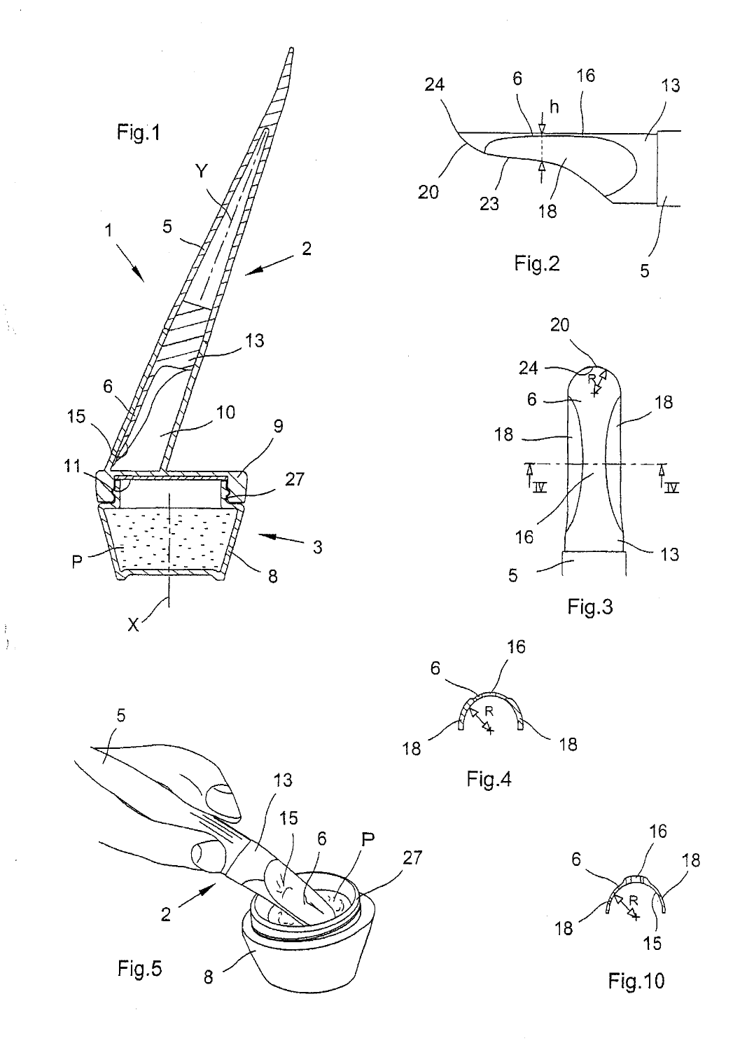

By way of example, the assembly 1 shown in FIG. 1 comprises an applicator 2 and a container 3 containing a nail varnish product, having, for example, a viscosity of greater than 0.6 Pa·s and/or thixotropic behavior and/or shear-thinning properties.

In the example under consideration, the applicator 2 comprises a handle shaft 5 and a flexible blade 6 serving for applying product P to the nails.

The container 3 comprises a body forming a reservoir 8 and a lid 9 having a chimney defining a housing 10 for receiving the blade 6 in storage configuration, as shown in FIG. 1.

The lid 9 may comprise a leaktightness member 11 such as a seal, a skirt or a cover, so as to obtain leaktight closure of the container 3 when not in use.

The housing 10 may have a longitudinal axis Y that makes an angle with the axis X of the reservoir body 8 so as to improve the stability of the assembly 1.

In the example under consideration, the blade 6 is connected to the hollow handle shaft 5 via a thicker, solid end portion 13 of circular cross section. The end portion 13 may be made so as to substantially block the aperture of the housing 10 when the applicator 2 is in place. The end portion 13 may be made of the same material as the flexible blade 6.

In one variant or alternate example illustrated in FIG. 21, the handle shaft 5 is made as a single component by moulding of material with the blade 6.

The blade 6 comprises, in the example under consideration, an upper wall 16 (FIGS. 2 and 3) defining the back of the blade and two wings 18 connecting to this wall 16, with the wall and the wings 18 together defining an application face 15 of generally concave shape. The application face 15 may define, in the illustrated examples, a developable surface.

As may be seen in FIG. 3, in the example under consideration, the distal edge 20 of the blade 6 is rounded in the top view, having at its apex 24 a radius of curvature r that is, for example, between 4 and 20 mm. The blade 6 may be symmetrical relative to a longitudinal midplane.

The height h of the wings 18 may gradually increase from the apex of the distal edge 20 towards the handle shaft 5, as may be seen in FIG. 2, passing, for example, through a point of inflection 23 that may be closer to the apex 24 than to the handle shaft 5.

The upper wall 16 may be made, as may be seen in FIG. 4, with a smaller thickness of material, so as to have increased flexibility.

In a median region of the blade located substantially midway between the apex 24 of the distal edge 20 and the handle shaft 5, the application face 15 may have, as illustrated in FIG. 4, an incurved shape, with a substantially constant radius of curvature R, for example between 4 and 20 mm, for example about 7 mm.

Where appropriate, the radii r and/or R may be infinite, the blade having, for example, a rectilinear distal edge perpendicular to its longitudinal axis and/or being flat.

The back of the blade may be substantially rectilinear and parallel to the longitudinal axis of the handle shaft 5, up to the thick portion 13. The application face 15 may be substantially cylindrical, with a generatrix parallel to the axis of the handle shaft.

The blade 6 may be made by moulding a thermoplastic material, especially a thermoplastic elastomer, the incurved shape coming from moulding. The blade 6 may optionally be reinforced at least at points with armature or a fibre filling.

The assembly 1 may be used in the following manner.

The user opens the container 3 and takes up using the applicator 2 the product P through the aperture of the body 8. Preferably, this body is made with a relatively wide neck 27, so as to facilitate this operation. The diameter of the neck 27 is, for example, greater than or equal to 2 cm.

For the uptake, the user can tap the product with the blade 6, the concavity of this blade being directed towards the product P. As a variant, the user can use the blade 6 like a spoon, its concavity being generally oriented upwards. Where appropriate, the user can wipe off the excess uptaken product on the edge of the neck 27.

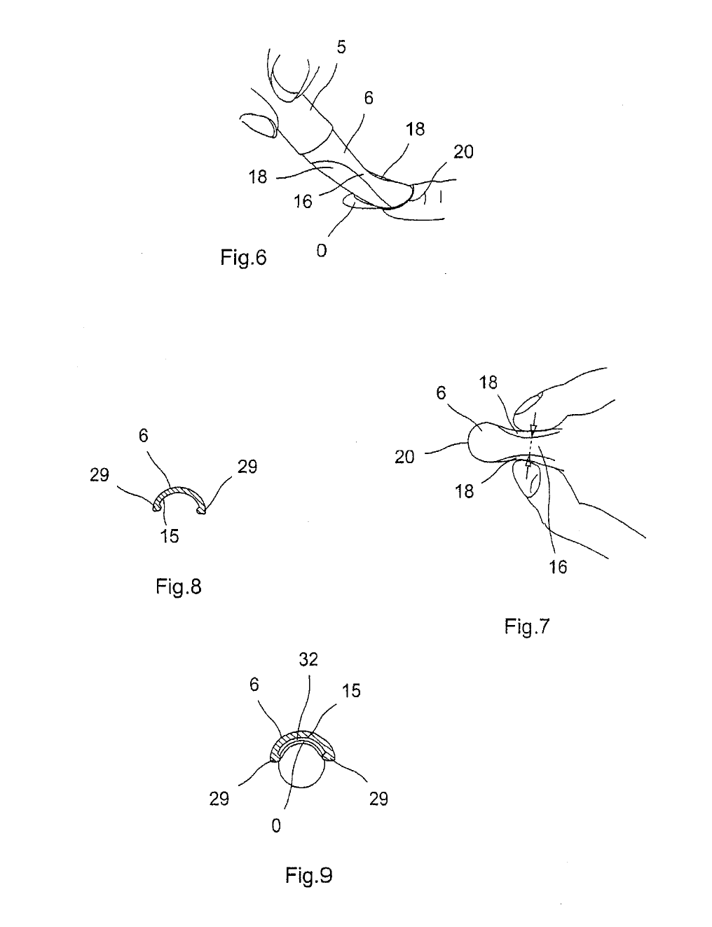

Next, the user can bring the application face 15 in contact with the nail O, as illustrated in FIG. 6, and apply the product by removing the distal edge 20 from the lunula in the direction of the free edge of the nail.

During application, the upper wall 16 may optionally become incurved, becoming concave on the side opposite the application face 15, and the wings 18 may become deformed to accompany the deformation of the upper wall 16.

Where appropriate, as illustrated in FIG. 7, the user can press with his or her fingers on the wings 18 so as to modify the curvature of the application face 15 and/or of the distal edge 20 in order especially to adapt the shape of the application face 15 and/or that of the distal edge 20 to the treated nail.

It is to be understood that the invention is not limited to the example described above.

By way of example, the blade 6 may comprise, on its longitudinal sides 8, ribs 29 projecting on the application face 15, as illustrated in FIG. 8.

These ribs 29 may extend parallel to the longitudinal axis of the blade 6.

The presence of the ribs 29 may make it possible to make between the application face 15 and the nail O, as illustrated in FIG. 9, a gap 32 of substantially uniform thickness, so as to more easily obtain a uniform deposit of product.

In the variant or alternate example illustrated in FIG. 10, the upper wall 16 is thicker than the wings 18.

The blade 6 may be made with very diverse shapes and the applicator 2 may or may not comprise a handle shaft to which the blade is connected.

The blade 6 may, for example, be initially made without incurvation, this incurvation possibly arising, where appropriate, from its mounting on an adapted handle shaft or from its handling by the user during application.

In the examples of FIGS. 11 to 14, the applicator does not comprise a separately manufactured handle shaft and the blade 6 is made as a single component by moulding of material with a handle part 36. The blade 6 may be made so as to be flat or incurved at rest.

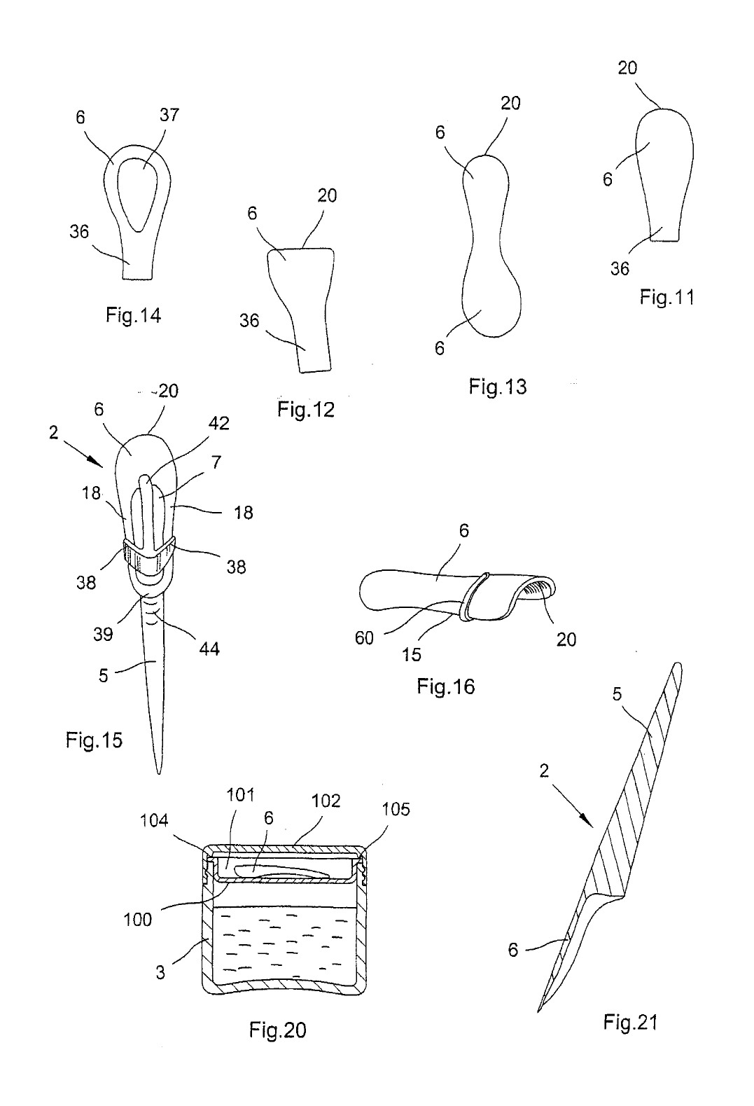

The distal edge 20 may be made with a more or less rounded, or even rectilinear, shape, as a function, for example, of the size of the nail to be made up, as illustrated in FIGS. 11 and 12. The blade 6 may especially have over at least a portion of its length, between its longitudinal sides, a thickness that increases on approaching the distal edge 20.

Further by way of example, the applicator 2 may comprise a blade 6 at each of its ends.

In the example of FIG. 13, the two blades 6 are made as a single component, for example by moulding of material, with different shapes so as to allow the treatment of a wider variety of nails. During use, one of the blades 6 serves as a handle part for the user.

The blade 6 may comprise openwork 37, as illustrated in FIG. 14, which may make it possible to increase the visibility of the nail during the application of makeup and may also make it possible to reduce the amount of product taken up by the applicator.

When the blade 6 is solidly attached to a handle shaft, the mounting of the blade 6 on the handle shaft may be performed so as to allow adjustment of the curvature of the application face 15.

In the example of FIG. 15, the handle shaft 5 comprises two lugs 38 that force the blade 6 to incurve. This blade may be similar to that of the example of FIG. 14, comprising openwork 37 into which the handle shaft 5 may be engaged, with the lugs 38 being applied, for example, from the outer side of the wings 18.

The blade 6 may bear against the handle shaft 5 via a proximal portion 39 opposite the distal edge 20.

The handle shaft 5 may comprise a finger 42 provided with a recess in which may engage the edge of the aperture of the blade 6.

The handle shaft 5 may comprise notches 44 that make it possible to draw the blade 2 onto the handle shaft 5 to a greater or lesser extent, these notches 44 being, for example, protrusions between which may be engaged the proximal portion 39 of the blade 6. The blade 6 is taut between the recess of the finger 42 and the selected notch 44.

In the example of FIG. 16, a control member 60 in the form of a ring engaged on the blade 6 allows, when moved thereon, the application face 15 to be incurved to a greater or lesser extent.

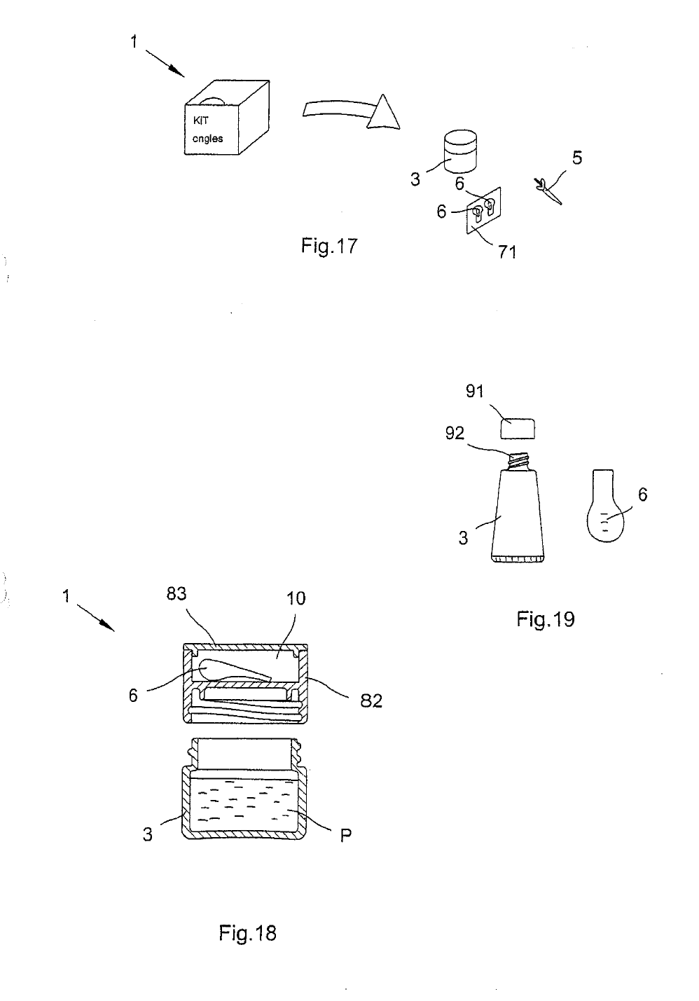

A plurality of blades 6 may be provided to the user, for example being precut in a sheet 71 of a flexible material, as illustrated in FIG. 17.

The user can detach a blade 6 and mount it on the handle shaft 5 before commencing the uptake of the product in the container.

The assembly 1 can include with several blades of identical or different shapes, optionally for single use, in the same packaging.

The assembly 1 shown in FIG. 18 comprises a container 3 in the form of a wide-necked jar.

The applicator 2 may be housed in a housing 10 of a closing capsule 82 of the container. This housing 10 may be closed by means of a lid 83.

In the example of FIG. 19, the product is contained in a container 3 in the form of a flexible tube provided with a stopper 91. To take up the product, the user squeezes the tube and places the outlet of the neck 92 of the tube above the application face, to deposit the product thereon.

In the example of FIG. 20, the blade 6 is contained in a housing 101 of a support 100 located inside the container 3. A lid 102 closes the container 3 and the housing 101. The support 100 may comprise a circular rim 105 that bears against the top end of the container 3. The lid may comprise a trim strip 104 that bears in a leaktight manner against the rim 105.

Many further modifications or alternate examples may be provided in accordance with the invention.

The applicator can include, for example, a portion or portions at least partially made with paper fibres, of a woven or a nonwoven, and further by way of example, can be impregnated with a polymer material that is compatible with the product P. The flexible blade may also be made of a metal, especially a material with shape memory, or even of a foam of low porosity or covered with a skin on the application face side, or of a paper or cardboard, optionally film-coated. The blade may comprise, for example, at least one armature, for example a metallic armature or a weave of synthetic reinforcing fibres.

By way of example, an applicator made in accordance with the invention can be used so as to produce a French manicure makeup, in which a composition of white color is deposited along the free edge of the nail. To this end, any of the applicators described above may be modified so as to have, when viewed from above, a concave free edge, as illustrated in FIG. 32. The blade may define a surface of developable application.

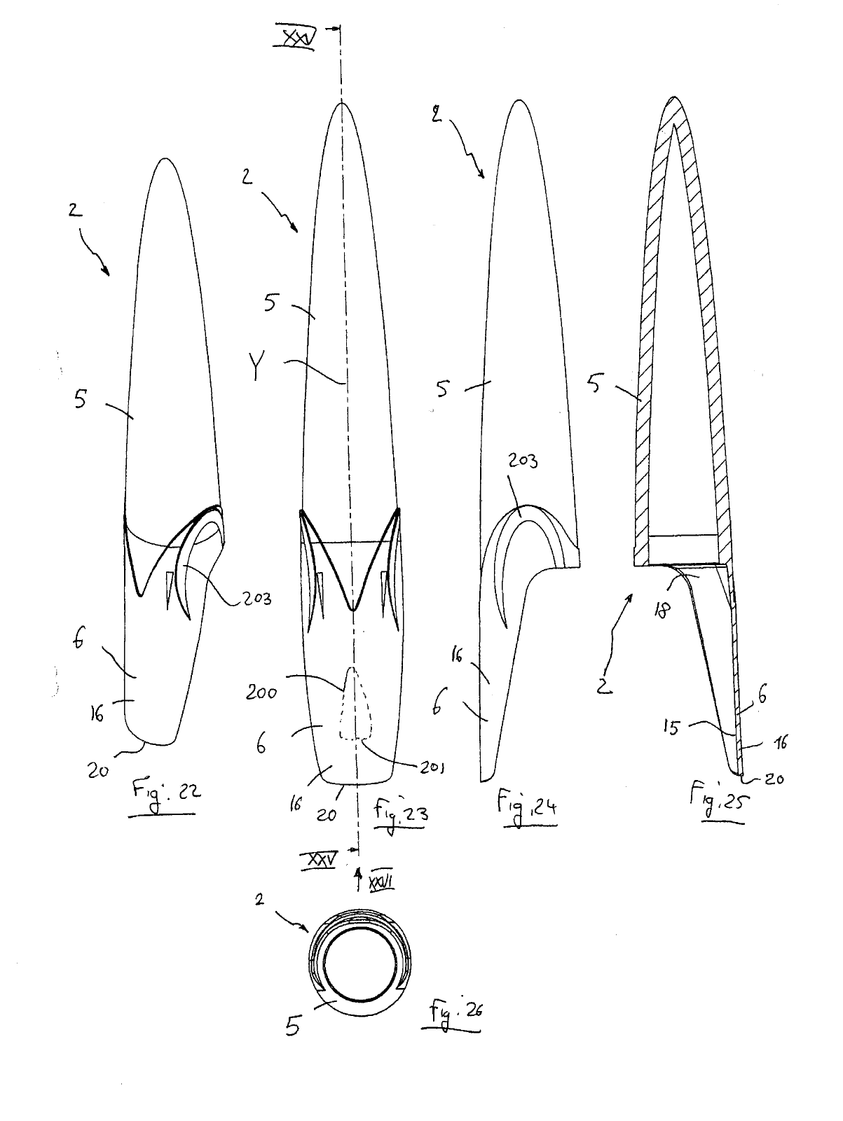

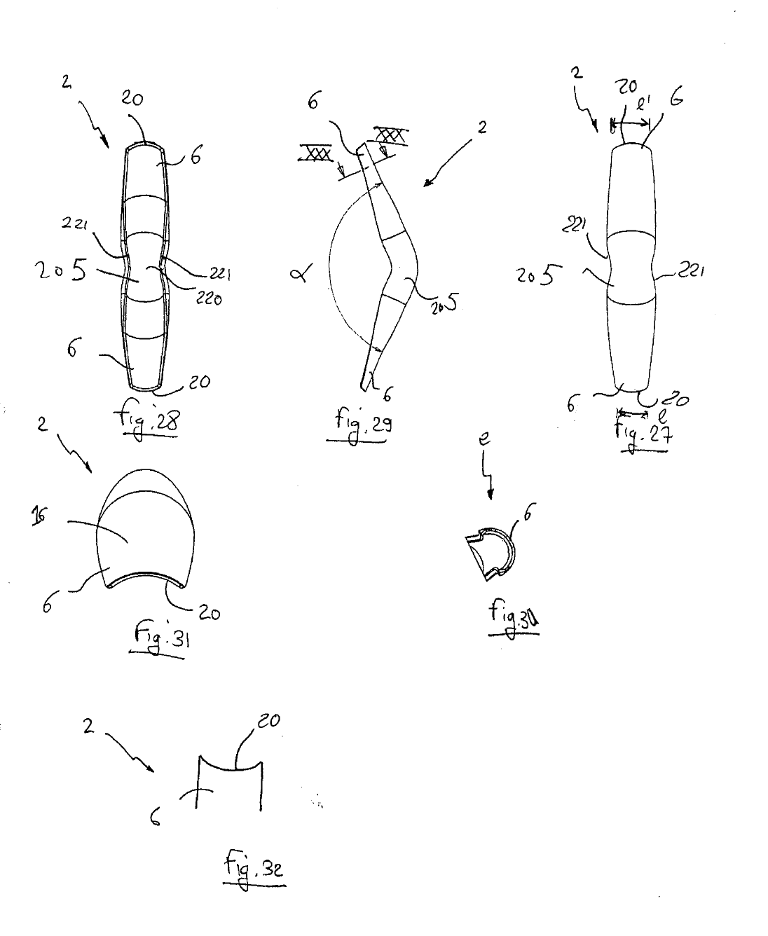

In the variant or alternate example illustrated in FIGS. 22 to 26, the blade is made as a single component with the hollow handle shaft 5 by moulding of a thermoplastic elastomer. This material is, for example, Santoprene® 82 8135med from the company Exxon Mobil, which is an SBS-SEBS mixture with a Shore hardness of 35 Shore A, or Engage® 8137 from the company Safic Alcan Dow, which is an ethylene octene with a Shore hardness of 57 Shore A.

Also by way of example, as illustrated in FIG. 23, the flexible blade 6 may be made with a void 200, which may increase the flexibility of the blade and especially allow it to become deformed on application when the user presses with its distal edge against the nail.

The void 200 may have, for example, a generally triangular shape, the base 201 of the triangle for example being rectilinear or curvilinear, for example circular when the distal edge of the blade is circular, optionally with the same center of curvature.

The blade 6 may be made with a lower thickness of material than the handle shaft 5, as is seen for example in FIG. 25, with the handle shaft 5 being made, for example, with a thickness of material at least twice that of the blade, the thickness of material of the blade being, for example, less than or equal to 1 mm, for example about 0.5 mm, whereas that of the handle shaft is greater than or equal to 1 mm.

Decorative ribs 203 may be produced on the handle shaft as illustrated in FIGS. 22 to 24.

The application face of the blade may define a surface of developable application, for example cylindrical or a portion of a cylinder.

In the example of FIGS. 28 to 31, the applicator comprises two flexible blades 6 at each of its ends, with these flexible blades being of different dimensions and/or of different profiles so as to be able to produce two different makeup results and/or to make up two nails of different sizes.

In the example under consideration, one of blades has a distal edge 20 that is shorter and of smaller radius of curvature than the other blade, so as to be able to apply the composition to smaller nails. The two flexible blades 6 are connected via a connecting part 205 that may serve as a handle means, the user also being able during application to hold the unused flexible blade to use it as a handle shaft.

The connecting part 205 may have a certain amount of deformability that allows the user to modify the angle α between the back of the flexible blades 6, the angle α being, for example, about 130° at rest.

The radius of curvature of the smaller blade 6 in the section plan of FIG. 30, measured on the outer surface of the blade, is, for example, between 6 and 7 mm, the thickness of the blade 6 being, for example, about 0.7 mm.

The length l, measured in the top view, of the distal edge of one of the blades is, for example, between 8 and 9 mm and the length l′ of the other distal edge is, for example, between 10 and 11 mm.

The connecting part 205 is, for example, formed with a back 220 and two side walls 221, as may be seen in FIG. 28, the walls 221 being inflected from each other in a median region.

Unless otherwise specified, terms such as “comprising,” “comprising one,” “including” should be understood as being synonymous with “comprising at least one.” Similarly, terms such as “comprising two” or “including two” mean “comprising at least two.” In addition, the terms “between” or “ranging from” means that the limits (or endpoints) are included.

### Process

A subject of the invention is also a process for making up the nails, which comprises lowering the viscosity of a nail varnish composition having a viscosity at 25° C. of at least 0.6 Pa·s, using an applicator as defined above or using any non-chemical action, simultaneously with or prior to the application of the composition to the nails.

According to one embodiment, the process comprises applying to the gelled composition (in its stored condition), located, e.g. in the container a non-chemical action, for example a mechanical stress, for example using an applicator as defined above, so as to fluidize and reduce the viscosity of the composition and enable its application to the nails. When the application ceases, the composition, after a rest time, regains its initial gel texture.

According to another embodiment, the process comprises taking up a sample of the composition (in its stored condition), and then in applying to the sample the non-chemical action, especially a mechanical stress, using an applicator as defined above, so as to fluidize the composition simultaneously with its application to the nails.

The non-chemical action may be chosen from thermal actions, for instance a source of heat, mechanical actions such as an object via which a mechanical stress or shear is applied to the composition, and combinations thereof. In particular, this object may be an applicator in the form of a fine brush, a spatula or a tip, and especially an applicator as described above.

Preferably, the non-chemical action is a mechanical action.

The nail varnish composition of the invention may be conditioned in a container delimiting at least one compartment, with the compartment being closed by a closing member.

The container may have any suitable form and may be at least partly made of a material such as glass. However, materials other than glass may be used, for instance thermoplastic products such as PP or PE, or a metal.

The invention is illustrated in greater detail in the examples that follow. Unless otherwise mentioned, the amounts are given as weight percentages relative to the total weight of the composition.

### Example 1

### Colored Nail Varnish

A nail varnish having the following composition (weight %) is prepared:

| | Example 1 |
|---|---|
| Nitrocellulose containing 30% isopropyl | 5.55 |
| alcohol (viscosity: E22-½ S) | |
| Nitrocellulose containing 30% isopropyl | 12.12 |
| alcohol (Idyl E27 from Bergerac) | |
| Nitrocellulose containing 30% isopropyl | 0.08 |
| alcohol (Azur E80 from Bergerac) | |
| Glycerophthalic alkyd resin esterified with | 15.50 |
| branched fatty acids, at 70% in ethyl | |
| acetate (Beckosol ODE 230 70E from Dainippon | |
| Ink and Chemicals) | |
| Isopropyl alcohol | 1.14 |
| Cyclopentadimethylsiloxane (DC245 Fluid from | 2 |
| Dow Corning) | |
| Polydimethylsiloxane 5 cSt (DC200 Fluid from | 0.44 |
| Dow Corning) | |
| Stearylbenzyldimethylammonium-modified | 2.56 |
| hectorite (Bentone 27 V from Elementis) | |
| Hydrophilic fumed silica (Aerosil 200 from | 0.46 |
| Degussa) | |
| Red 7 lake | 0.02 |
| Titanium oxide mica (Timiron Super Silk | 0.55 |
| MP 1005 from Merck) | |
| Titanium oxide mica (Flamenco Red 420 C from | 0.2 |
| Engelhard) | |
| Bismuth oxychloride | 0.78 |
| Ethyl acetate | 19.27 |
| Acetyl tributyl citrate | 7.54 |
| Butyl acetate | qs 100 |
| Citric acid monohydrate | 0.1 |

The composition has a viscosity at 25° C. of 0.820 Pa·s and is conditioned in a jar.

Mechanical agitation or stress is applied to the composition using an applicator comprising a flexible blade defining an application face, as described in FIG. 6. The composition fluidizes and the fluidized composition is then applied to the nails using the applicator. A glossy film that covers the nails well is obtained.

After a few minutes, the nail varnish regains its initial texture (viscosity close to the initial viscosity).

### Example 2

### Colorless Nail Varnish

| | Example 2 |
|---|---|
| Nitrocellulose containing 30% isopropyl | 5.2 |
| alcohol (viscosity: E22-½ S) | |
| Nitrocellulose containing 30% isopropyl | 13.70 |
| alcohol (Idyl E27 from Bergerac) | |
| Glycerophthalic alkyd resin esterified with | 16.19 |
| branched fatty acids, at 70% in ethyl | |
| acetate (Beckosol ODE 230 70E from Dainippon | |
| Ink and Chemicals) | |
| Isopropyl alcohol | 0.99 |
| Polydimethylsiloxane 5 cSt (DC200 Fluid from | 0.5 |
| Dow Corning) | |
| Stearylbenzyldimethylammonium-modified | 2.8 |
| hectorite (Bentone 27 V from Elementis) | |
| Hydrophilic fumed silica (Aerosil 200 from | 0.526 |
| Degussa) | |
| Ethyl acetate | 19.94 |
| Acetyl tributyl citrate | 7.96 |
| Butyl acetate | qs 100 |
| Citric acid monohydrate | 0.1 |

### Example 3

### Nail Varnish

a) Synthesis of a pentaerythrityl benzoate/isophthalate/isostearate polycondensate

227.5 g of benzoic acid, 72.8 g of isostearic acid and 118.3 g of pentaerythritol are placed in a reactor equipped with a mechanical stirrer, an argon inlet and a distillation system, and the reactor is then heated gradually, under a gentle stream of argon, to 110-130° C. to obtain a homogeneous solution. The temperature is then gradually raised to 180° C. and maintained for about 2 hours. The temperature is again raised to 220° C. and maintained until an acid number of less than or equal to 1 is obtained, which takes about 18 hours. The mixture is cooled to a temperature of between 100 and 130° C., 91 g of isophthalic acid are then introduced and the mixture is gradually heated again at 220° C. for about 11 hours.

430 g of pentaerythrityl benzoate/isophthalate/isostearate polycondensate are thus obtained in the form of a thick oil that solidifies at room temperature.

The polycondensate has the following characteristics:
acid number=12.7
hydroxyl number=49
η_{110° C.}=25.4 poises (i.e. 2540 mPa·s)
ratio between the number of moles of aromatic monocarboxylic acid and the number of moles of non-aromatic monocarboxylic acid: 7.28.

420 g of the polycondensate obtained above are taken and heated to 100-120° C., 180 g of butyl acetate are poured in slowly with stirring and the mixture is then clarified by hot filtration through a No. 2 sinter.

After cooling to room temperature, 600 g of polycondensate at 70% in butyl acetate are obtained, in the form of a pale yellow viscous liquid with a viscosity at 25° C. of about 800 centipoises (mPa·s).

### b) The Following Nail Varnish is Prepared:

| | |
|---|---|
| Nitrocellulose containing 30% isopropyl | 4.84 |
| alcohol (viscosity: E22-½ S) | |
| Nitrocellulose containing 30% isopropyl | 12 |
| alcohol (Idyl E27 from Bergerac) | |
| Nitrocellulose containing 30% isopropyl | 0.08 |
| alcohol (Azur E80 from Bergerac) | |
| Glycerophthalic alkyd resin esterified with | 2.45 |
| branched fatty acids, at 70% in ethyl | |
| acetate (Beckosol ODE 230 70E from Dainippon | |
| Ink and Chemicals) | |
| Solution containing 70% solids of the | 11.49 |
| polymer synthesized in a) in butyl acetate | |
| Isopropyl alcohol | 1.4 |
| Cyclopentadimethylsiloxane (DC245 Fluid from | 2 |
| Dow Corning) | |
| Polydimethylsiloxane 5 cSt (DC200 Fluid from | 0.44 |
| Dow Corning) | |
| Stearylbenzyldimethylammonium-modified | 3.19 |
| hectorite (Bentone 27 V from Elementis) | |
| Hydrophilic fumed silica (Aerosil 200 from | 0.37 |
| Degussa) | |
| Red 7 lake | 0.02 |
| Titanium oxide mica (Timiron Super Silk | 0.55 |
| MP 1005 from Merck) | |
| Titanium oxide mica (Flamenco Red 420 C from | 0.2 |
| Engelhard) | |
| Bismuth oxychloride | 0.78 |
| Ethyl acetate | 15 |
| Acetyl tributyl citrate | 7.37 |
| Butyl acetate | qs 100 |
| Citric acid monohydrate | 0.13 |

The composition has a viscosity at 25° C., measured using a Rheomat 180 viscometer, of 0.820 Pa·s.

Mechanical stirring is applied to the composition using an applicator as defined in the present application, the composition becomes fluid and the fluidized composition is then applied to the nails using the applicator. A glossy film that covers the nails well, without striations, is obtained.

After a few minutes, the nail varnish regains its initial texture (viscosity close to the initial viscosity).

The thixotropic behavior of the composition is evaluated via the viscosity measurements of the composition according to the protocol described hereinabove.

The composition has a viscosity, as measured in step e), of 45 Pa·s.

It has thixotropic behavior preferably with the viscosity difference (viscosity measured during step c) at a shear rate of 1 s⁻¹−viscosity measured during step e) at a shear rate of 1 s⁻¹) of about 100 Pa·s.

This composition has a plateau stiffness modulus Gp of between 1000 and 3000 Pa, of about 2000 Pa, an elasticity δₚ ranging for example from 20°, and a flow threshold τ_{c} of 100 Pa.

c) The Following are Prepared:
a nail varnish of gelled texture (Example 4 according to the invention) with a viscosity, measured according to the protocol indicated in the description, of greater than 0.6 Pa·s, and
a nail varnish in liquid form according to the prior art (comparative Example 5) whose viscosity is not measurable according to the protocol indicated hereinabove.

The varnishes have the following composition (weight %):

| | Example 4 | Example 5 |
|---|---|---|
| | (invention) | (comparative) |
| Solution containing 70% solids | 12.18 | — |
| of the polymer of Example 1 in | | |
| butyl acetate | | |
| Nitrocellulose containing 30% | 4.18 | 12.37 |
| isopropyl alcohol (viscosity: | | |
| E22-½ S) | | |
| Nitrocellulose containing 30% | 12.17 | — |
| isopropyl alcohol (Idyl E27 | | |
| from Bergerac) | | |
| Nitrocellulose containing 30% | — | — |
| isopropyl alcohol (viscosity: | | |
| E28- 1/8 S) | | |
| Nitrocellulose containing 30% | 0.004 | 1.63 |
| isopropyl alcohol (Azur E80 | | |
| from Bergerac) | | |
| Glycerophthalic alkyd resin | 1.42 | 15.53 |
| esterified with branched fatty | | |
| acids, at 70% in ethyl acetate | | |
| (Beckosol ODE 230 70E from | | |
| Dainippon Ink and Chemicals) | | |
| Cyclopentadimethylsiloxane | 2 | — |
| (DC245 Fluid from Dow Corning) | | |
| Polydimethylsiloxane 5 cSt | 0.47 | — |
| (DC200 Fluid from Dow Corning) | | |
| Stearylbenzyldimethylammonium- | 3.3 | 1.23 |
| modified hectorite (Bentone 27 V | | |
| from Elementis) | | |
| Hydrophilic fumed silica | 0.4 | — |
| (Aerosil 200 from Degussa) | | |
| Glass particles coated with | 1.5 | 1.5 |
| titanium oxide (Metashine | | |
| MC 1040RY from Nippon Sheet | | |
| Glass | | |
| Glass particles coated with | 1.5 | 1.5 |
| titanium oxide (Metashine | | |
| MC 1040RS from Nippon Sheet | | |
| Glass | | |
| Titanium oxide mica (Timiron | 0.8 | 0.8 |
| Super Gold from Merck) | | |
| Red lake 34 | 0.0045 | 0.0045 |
| Red lake 6 | 0.005 | 0.005 |
| Ferric blue | 0.00009 | 0.00009 |
| Isopropyl alcohol | 1.23 | 3.58 |
| Ethyl acetate | 14.27 | 22.51 |
| Acetyl tributyl citrate | 7.31 | 1.73 |
| Butyl acetate | qs 100 | qs 100 |
| N-Ethyl-O,P-toluenesulfonamide | — | 4.85 |
| Citric acid monohydrate | 0.13 | 0.05 |

The stability of the formulations over time may be evaluated by placing a bottle of each formulation in an oven at 45° C. for 2 months, and then by visually observing the change in homogeneity of the formulations over time.

The observer assigns a grade ranging for example from 0 to 5 after 2 months at 45° C. (0=maximum sedimentation; 5=no sedimentation).

The stability of the nail varnish according to the invention did not vary after 2 months at 45° C. (no sedimentation of the pigments), whereas the conventional nail varnish in liquid form shows sedimentation of the pigments at the bottom of the bottle and a non-uniform color.

Obviously, numerous modifications and variations of the present invention are possible in light of the above teachings. It is therefore to be understood that within the scope of the appended claims, the invention may be practiced otherwise than as specifically described herein. It is also to be realized that the compositions described above, and those that make up a part of the original assembly, process, etc. claims appended hereto, themselves are a part of the present invention.

1. A conditioning and application assembly comprising:
a container containing a nail varnish composition comprising a cosmetically acceptable medium and at least one thixotropic thickener, the composition having a viscosity at 25° C. of at least 0.6 Pa·s, and
an applicator for taking up the product contained in the container and applying it to the nails, comprising a flexible blade defining an application face.

2. An assembly according to claim 1, wherein the composition has a viscosity ranging from 0.6 to 20 Pa·s.

3. An assembly according to claim 2, wherein the composition has a viscosity of from 0.7 to 15 Pa·s.

4. An assembly according to claim 3, wherein the composition has a viscosity from 0.75 to 10 Pa·s.

5. A conditioning and application assembly comprising:
a container containing a nail varnish composition comprising a cosmetically acceptable medium and at least one thixotropic thickener, the composition having a thixotropic nature such that application of a shear stress results in a change in viscosity of at least 1 Pa·s, and
an applicator for taking up the product contained in the container and applying it to the nails, comprising a flexible blade defining an application face.

6. An assembly according to claim 5, wherein the difference in viscosity (viscosity measured during step c)−viscosity measured during step e)) is greater than or equal to 10 Pa·s, better still at least 20 Pa·s, even better still at least 30 Pa·s and preferentially at least 40 Pa·s.

7. An assembly according to claim 5, wherein the change in viscosity ranges from 40 to 200 Pa·s.

8. A conditioning and application assembly comprising:
a container containing a nail varnish composition comprising a cosmetically acceptable medium and at least one thixotropic thickener, the composition having a plateau stiffness modulus Gp of greater than 100 Pa, and
an applicator for taking up the product contained in the container and applying it to the nails, comprising a flexible blade defining an application face.

9. An assembly according to claim 8, wherein the plateau stiffness modulus Gp ranges from 100 to 2×10⁶ Pa·s.

10. An assembly according to claim 9, wherein the plateau stiffness modules Gp ranges from 5×10² to 10⁴ Pa·s.

11. An assembly according to claim 10, wherein the plateau stiffness Gp ranges from 800 to 4000 Pa·s.

12. An assembly according to claim 9, wherein the plateau stiffness Gp ranges from 1000 to 3000 Pa·s.

13. An assembly according to claim 8, wherein the thixotropic thickener is chosen from hydrophilic or organophilic clays, hydrophilic or hydrophobic fumed silicas, and elastomeric organopolysiloxanes, and mixtures thereof.

14. An assembly according to claim 8, wherein the thixotropic thickener is chosen from organophilic modified clays.

15. An assembly according to claim 14, wherein the thixotropic thickener includes hectorite modified with benzyldimethylammonium stearate.

16. An assembly according to claim 8, wherein the thixotropic thickener also comprises a hydrophobic fumed silica.

17. An assembly according to claim 8, wherein the thixotropic thickener is present in a content ranging for example from 1.7% to 15% by weight relative to the total weight of the composition.

18. An assembly according to claim 17, wherein the thixotropic thickener is present in a range from 2% to 10% by weight.

19. An assembly according to claim 17, wherein the thixotropic thickener is present in a range of from 3.5% to 7.5% by weight.

20. An assembly according to claim 1, wherein the flexible blade is incurved and defines a generally concave application face.

21. An assembly according to claim 1, wherein the flexible blade includes opposite bearing surfaces for the fingers, so as to allow the user to modify the curvature thereof.

22. An assembly according to claim 1, wherein the flexible blade has a non-uniform thickness.

23. An assembly according to claim 22, the blade comprising a back located between wings that are thicker than the back.

24. An assembly according to claim 1, wherein the flexible blade has a convex distal edge when viewed from above.

25. An assembly according to claim 1, the applicator comprising a member for controlling the curvature of the application face.

26. An assembly according to claim 1, wherein the composition comprises at least one coloring agent chosen from magnetic substances, diffractive pigments, interference pigments and reflective particles, and mixtures thereof, the coloring agent being present in a content of greater than or equal to 2% by weight relative to the total weight of the composition.

27. An assembly according to claim 26, wherein the diffractive pigments are chosen from:
monolayer pigments comprising a reflective material chosen from metals and alloys thereof,
pigments with a multilayer structure comprising a layer of a reflective material chosen from metals and alloys thereof and also from non-metallic reflective materials, coated on at least one side with a layer of a dielectric material,
pigments composed of a preformed dielectric or ceramic material such as a natural mineral leaflet, or synthetic leaflets,
and mixtures thereof.

28. An assembly according to claim 26, wherein the interference pigments are chosen from nacres, reflective interference particles and goniochromatic pigments, and mixtures thereof.

29. An assembly according to claim 26, wherein the reflective particles are chosen from:
metal oxides, especially titanium or iron oxides obtained synthetically,
multilayer structures comprising a natural or synthetic substrate, at least partially coated with at least one layer of a reflective material, especially of at least one metal or metallic material.

30. An assembly according to claim 26, wherein the magnetic substances or particles comprise a magnetic material chosen from the group constituted by: iron, nickel, cobalt, and alloys and oxides thereof, especially Fe₃O₄.

31. An assembly according to one of claim 26, wherein the coloring agent is present in a content of greater than or equal to 2.5% by weight relative to the total weight of the composition.

32. An assembly according to claim 31, wherein the coloring agent is present in an amount equal to or greater than 4% by weight.

33. A process for making up nails, comprising the following steps:
taking up a nail varnish having a viscosity at 25° C. of at least 0.6 Pa·s in a container, using a blade of an applicator, and
applying the product using the application face of the blade.

34. A process for making up the nails, which comprises lowering the viscosity of a nail varnish composition having a viscosity at 25° C. of at least 0.6 Pa·s, using an applicator, simultaneously with or prior to the application of the composition to the nails.

35. A process according to claim 34, in which, before or after taking up the product, the user modifies the curvature of the blade to that of the treated nail.

36. A process according to claim 34, wherein the nail varnish composition has a viscosity which is reduced by at least 1 Pa·s upon application of mechanical stress by said applicator.
